(19) United States
(12) Patent Application Publication
(10) Pub. No.: US 2005/0233298 A1
(43) Pub. Date: Oct. 20, 2005

(54) Portable and other consumer storage for biological material

(76) Inventor: Lewis E. Farsedakis, Vienna, VA (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/823,627

(22) Filed: Apr. 14, 2004

(51) Int. Cl. A01N 1/00; C12M 3/00

(52) U.S. Cl. 435/1.1; 435/284.1; 435/307.1

(57) ABSTRACT

Portable and other consumer storage is provided for stem cells and other valuable biological material. The consumer's reliance on a stem cell bank is removed.

### BACKGROUND OF THE INVENTION

Conventionally, placental and umbilical cord material has been collected and stored in certain ways and for certain purposes, see, e.g., U.S. Pat. No. 6,238,907 (“Container for storing and examining placentas”); U.S. Pat. No. 5,372,581 (“Method and apparatus for placental blood collection”); U.S. Pat. No. 5,298,020 (“Neonatal autotransfusion apparatus and method”); U.S. Pat. No. 5,993,398 (“Computer-based mixed-use registry of placental and umbilical cord stem cells”); U.S. patent application No. 20010054429 (“Methods for collecting cord blood and related devices”); U.S. Pat. Nos. 6,605,275, 6,569,427 and 6,461,645 (“Isolation and preservation of fetal and neonatal hematopoietic stem and progenitor cells of the blood”). As one example, placental material had long been contained temporarily as a prelude to its being disposed of as medical waste. As another, quite different, example, medical and genetic applications of cord stem cells have become well-known.

Recently scientists have recognized that umbilical cord blood of a newborn human child can be stored and used medically or clinically later in that same child's life, for the benefit of that same child. There have begun to emerge certain “banking” systems, such as businesses that collect and preserve a baby's umbilical cord blood, which service has been said to conventionally cost about $1,500 for collection and $95/year for storage by the company. See Kline, “Whose blood is it anyway?,” in Scientific American, special issue 2003, pages 22-27. See also U.S. patent application No. 20030014285, published Jan. 16, 2003, by R. Daniel, “No pay annuity method;” U.S. Pat. No. 5,993,387 (“Computer-based mixed-use registry of placental and umbilical cord stem cells;” U.S. Pat. No. 6,640,211 (“Genetic profiling and banking system and method”); and www.lifebankusa.com.

It also may be considered that, in recent times, political and other controversies have emerged, certainly in the United States, regarding such biological materials as stem cells. Persons (such as parents) wanting to “bank” cord blood or umbilical cord materials cannot be certain how laws and politics may evolve or change, and whether the “bank” may fail to perform as contracted. Moreover, regardless of political and legal developments, whether the “bank” will perform as contracted, throughout a long period of time, can be a concern.

### SUMMARY OF THE INVENTION

Such a business providing a banking service has a different motivation for continuing to provide banking services as agreed, at the proper level to preserve and maintain valuable biological materials, than does a consumer entrusting valuable biological materials to the bank. Even if the bank is performing perfectly competently, a consumer having let those valuable biological materials out of his or her immediate direct control may have worry or anxiety about the condition of the stored materials. The present invention alleviates a consumer's reliance on others (such as a stem cell bank, etc.) and increases the amount of control and influence that an individual and/or the family of the individual may have over his, her or their health, appearance, and/or medical condition.

The present invention, in a particularly preferred embodiment, provides for a system for a customer to maintain physical control of valuable biological material, with the system comprising at least one of: A) direct consumer responsibility for and/or direct consumer supervision of collection of a source of valuable biological material of an infant or young child related to the consumer; and/or B) direct consumer storage of the source of valuable biological material and/or a quantity of valuable biological material derived from the source of valuable biological material.

The present invention provides for storage devices, storage systems, and storage methods, for accomplishing “consumer storage” of valuable biological material. The desirable “consumer storage” may be by an individual (such as by a parent of valuable biological material of a child (especially, in a preferred embodiment, a newborn child) of the parent. In the present invention, the “consumer” is considered to be some individual (such as a parent) having a familial and/or genetic relationship to the valuable biological material (such as a mother, father, grandparent, etc.), or another cognizable valid relationship to the valuable biological material (such as a guardian of a child to whom was physically attached or whom was physically part of the valuable biological material, a legal adoptive parent of such a child, etc.). Particularly advantageous features provided, in especially preferred embodiments, for the inventive “consumer storage” are one or more of: (A) portability, such as being easily carried by one individual; (B) useability for self-storage by at least one parent of umbilical cord material of a child of the parent.

The invention provides, in one preferred embodiment a system for consumer control of valuable biological material, comprising at least the steps of: A) collection of a source of valuable biological material of an infant or young child; B) processing the source of valuable biological material into a quantity of valuable biological material suitable for consumer storage. Optionally, there may be further included a step C) of consumer storage of the quantity of valuable biological material from step B) (such as, for example, the quantity of valuable biological material under consumer storage being stored under direct physical custody and control of at least one parent, or parent's designated guardian or custodian, of the infant or young child from whom collection was undertaken, and/or the quantity of valuable biological material suitable for consumer storage being in a container labeled with identifying information and/or instructions.)

In another particularly preferred embodiment, the present invention provides a storage device for consumer storage of a quantity of valuable biological material (such as valuable biological material from, e.g., a human umbilical cord, etc.) of a human child, comprising: a compartment into which may be received the valuable biological material having certain desired biological features; a fastening system for securing the compartment containing the valuable biological material to provide a secured, closed compartment housing the valuable biological material; and a preservation system for maintaining the certain desired biological features of the valuable biological material in the secured, closed compartment. Optionally, such an inventive device may be one or more of: (A) portable and easily carried by one individual; (B) useable for self-storage by at least one parent of umbilical cord material of a child of the parent.

Particularly preferred examples of a value of the valuable biological material includes, e.g., for actually or potentially treating or alleviating a medical condition, disease, disorder or problem of the same child; a sibling; and/or another family member; for actually or potentially effecting a cosmetic result for the same child; a sibling; and/or another family member.

There also may be mentioned exemplary methods, systems, and storage devices according to the present invention, in which the child from whom the valuable biological material came was a newborn human, and wherein the valuable biological material has been stored and preserved since at least one of: infancy of the child; the child being a toddler; the child becoming a teenager; the child surviving teenage years; the child surviving his or her twenties; the child surviving his or her thirties; the child himself or herself having a baby.

### SUMMARY OF THE FIGURES

FIG. 1 is a flow chart of an exemplary consumer-storage method according to the present invention.

FIG. 2 is a flow chart of an exemplary consumer-involved method according to the present invention, in which method a consumer prepares for collection of a source of valuable biological material.

### DETAILED DESCRIPTION OF PREFERRED EMBODIMENT

The present invention is directed to the self-storage and/or the portable storage or biological material that is “valuable” with reference to that same individual who was the source of the biological material. Examples of a value that makes biological material “valuable” are, e.g., for actually or potentially treating or alleviating a medical condition, disease, disorder or problem; for actually or potentially effecting a cosmetic result; etc. For example, stem cells have been recognized as having such value. The invention is directed in a most preferred embodiment to an individual's re-use, in later life, of his or her own biological material, such as in later life using his or her own genetic material, own stem cells, etc. In other embodiments, the re-use of stored biological material is for the benefit of another person, such as a sibling or other family, etc.

Examples of sources of the “valuable biological material” that is mentioned are, for example, placenta or placental material, umbilical cord, cord blood, any other source of stem cells, etc. Most preferred as a source of “valuable biological material” according to the present invention is a source that is non-invasive, such as a placenta or umbilical cord. The “valuable biological material” that is stored or preserved according to the present invention may be derived from or removed from the above-mentioned sources (e.g., from the placenta, placental material, umbilical cord, cord blood etc.) and may be all, or less than all of the source. Preferably, the valuable biological material is less than all of the source material, i.e., the source material has been treated to remove contaminants. It will be appreciated that a source of valuable biological material may contain, in addition to the valuable biological material, contaminants that are not cells of the child, with examples of contaminants being bacteria or other living organisms. For example, in the mother's birth canal (through which a baby being delivered usually passes) are routinely present bacteria and other organisms which are not cells of the mother, and which a baby being born encounters. Thus, there may be present in the source material biological material which is not genetically the child's. In a typical adult human, ˜90% (by number) of the cells living in the human may be non-self cells, i.e., bacteria, parasites, etc. living within the individual, with the non-self cells being (generally) relatively-small compared to the cells with the individual's genetic material, but comparatively numerous.

In the present invention, the source (e.g., umbilical cord, cord blood, placenta, etc.) of the valuable biological material may, for example, be collected by a physician, nurse or other professional attending the birth, by a family member, other authorized individual, etc. Most preferably, the source of the valuable biological material is collected as early and in as sterile a manner as possible. Collection of such source biological materials for processing to obtain stem cells may be accomplished according to well-known and established procedures.

It will be appreciated that in the present invention, at the earliest reasonably practicable time and before long-term storage, preferably the source is processed, such as by treatment to remove any contaminant and/or undesired component that would destroy the valuable biological material. Even more preferably, before long-term storage there is removed from the source anything which is not the genetic material of the child. However, in such a case, there still remains both valuable biological material and non-valuable biological material of the child in a sample for long-term storage. Thus, most preferably, before long-term storage there is additionally removed cells or tissues of the child which are not valuable, so that in the sample for long-term storage there remains only valuable biological material of the child. That is, in the most preferred embodiment, what is being stored long-term is valuable biological material of a child, without also storing contaminants, bacteria, microorganisms, unwanted decaying tissue, etc. Processing of the source biological materials for reducing the amount of unwanted material in the sample while retaining the valuable biological material may be by using well-known and established procedures. While the preferred and most preferred embodiments for long-term storage have thus been mentioned, it will be appreciated that the invention does not prohibit, and permits, the sample to-be-stored-long-term to include any component which does not destroy the valuable biological material.

In a most preferred embodiment of the invention, the source (such as the umbilical cord, placenta, etc.) is processed to provide a consumer-maintainable quantity of valuable biological material, such as a consumer-maintainable quantity of stem cells. For example, a consumer-maintainable quantity of stem cells of an infant child is prepared as a sample for at-home maintaining by at least one parent of the infant child. It will be appreciated that the entire source need not be processed into the consumer-maintainable quantity of valuable biological material. Optionally, the source may be processed into more than one quantity of valuable biological material, of which at least one quantity may be made into a sample for at-home maintaining and wherein at least one quantity may be made into a sample for non-consumer maintaining, such as at a stem cell bank or a genetic bank.

It will be appreciated that in the present invention, there may be used more than one storage compartment at different times, such as, e.g., upon collection using a short-term storage container for placental material, blood, umbilical cord etc. and then, later, a long-term storage container for a sample extracted or derived from treated placental material, blood, umbilical cord, etc.

In any event, the conditions (temperature, pH, fluids or nutrients, etc.) at which are kept the valuable biological material (whether it is contained within a source material, or is in treated or untreated (contaminated) form) should at all times be such as to preserve and maintain the valuable biological material as to remain useable. The minimum conditions under which the valuable biological material should be maintained are those such that the individual from whom the valuable biological material was derived may at a later time (such as in future months, years, and decades) enjoy a therapeutic, medical, cosmetic or other biological use with regard to his or her own body. More preferably, the conditions under which the valuable biological material should be maintained are those such that the individual from whom the valuable biological material was derived may at a later time enjoy any or all known therapeutic, medical, cosmetic and other biological uses with regard to his or her own body, that is, the valuable biological material is kept as viable or living as possible.

For example, in one embodiment, there may be provided a system or device comprising: a compartment into which may be received a quantity of valuable biological material having certain desired biological features; a fastening system for securing the compartment containing the quantity of valuable biological material to provide a secured, closed compartment housing the quantity of valuable biological material (such as valuable biological material from a human umbilical cord, etc.); and a preservation system for maintaining the certain desired biological features of the quantity of valuable biological material in the secured, closed compartment.

It will be appreciated that the most preferred embodiment of the invention calls for storage permanency, to the longest time possible (whether that time is measured in absolute time, such as years, or measured with relation to the life of the child producing the valuable biological material), of the valuable biological material from the child, so that the same child may have benefit of his or her own valuable biological material later in life, whether for treating or alleviating a medical disease, disorder, ailment (however serious or minor), for cosmetic reasons, for reasons presently contemplated or medical procedures that may exist at some time in the future but do not yet exist today. For example, the valuable biological material from a human newborn child may be stored and preserved for use by or for that same child, e.g., when the child is an infant, toddler, teenager, adult, of any age (such as post-twenties, post-thirties), at any time (including, but not limited to, when the child himself or herself is having or has had a baby). The term “child” (from whom the valuable biological material comes) is used herein with a broad meaning, to refer to that individual throughout his or her entire lifetime.

In a particularly preferred embodiment of the invention, the portability of the storage compartment is highly controllable by the consumer, such as, for example, by providing a two-part system comprising, first, the storage compartment housing the child's valuable biological material is readily portable, light-weight and easily carried when the consumer wants to remove and transport the storage compartment, and, second, comprising a securing base into which the storage compartment, when the consumer does not specifically at that moment desire to transport it, may be inserted and locked. Thus, there may be formed a configuration of an immobilized storage compartment according to the invention locked to a securing base. Advantages of such a portable storage compartment include, e.g., minimizing risk of theft and inadvertent disposal of the storage compartment, etc.

It will be appreciated that biological materials are not generally as freely transportable as most non-biological articles. That is, various laws, rules, regulations and policies apply, in the U.S. and elsewhere, to shipping and transportation of biological materials. Such laws, rules, regulations and policies are not necessarily static, and are probably highly likely to be changing in the coming years. In a most preferred embodiment, a portable storage device or system according to the present invention optionally may be used in combination with a computerized system (such as, e.g., an Internet-accessible system) that may be accessed by the consumer wanting to transport the portable storage device or system to a destination (such as a different country) to receive a short summary, in layperson format, of current applicable law, regulations and the like applicable to the desired transportation of the portable storage device or system to that destination. For example, such a computerized system used with a portable storage device or system would alleviate the risk of a consumer inadvertently transporting the storage compartment to a destination where such activity would or may be considered illegal or prohibited.

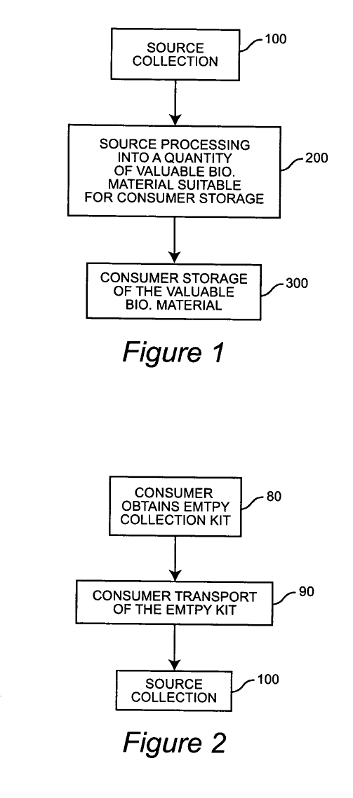

An exemplary inventive method is described with reference to FIG. 1. In this example according to the inventive, source collection 100 is performed, to provide a source (with examples of a source being a placenta, an umbilical cord, cord blood, etc.) of valuable biological material (with examples of valuable biological material being stem cells, etc.). Source processing 200 is performed to provide a quantity of valuable biological material (such as stem cells) suitable for consumer storage (with examples of consumer storage being storage in a family's home, portable storage in a carry-case, etc.).

With further reference to FIG. 1, the quantity of valuable biological material suitable for consumer storage may undergo consumer storage 300 of the valuable biological material. For example, parents of the child from whom was derived the valuable biological material (such as stem cells) may maintain the consumer-maintainable sample in their home. Optionally, for the consumer-maintainable sample, there may be provided an at-home apparatus and/or container for maintaining the biological viability of the quantity of valuable biological material.

The present invention further optionally makes provision for the consumer (such as an expectant parent, etc.) to be actively involved on the consumer's end for himself and/or herself bringing to the delivery room or other point source collection a suitable empty collection kit (such as, e.g., an empty collection kit offered by an Ob/Gyn doctor to his patient for purchase, etc.) for collecting the source of the valuable biological material (such as umbilical cord, placenta, etc.). For example, with reference to FIG. 2, a consumer may obtain 80 an empty collection kit followed by consumer transport 90 of the empty kit to the source collection 100, with the source collection 100 most preferably being performed by a medical professional who is already participating in or otherwise present during or soon after the birth of the baby. The person (such as, for example, an expectant mother) who obtains 80 (such as, e.g., purchasing from a physician or other seller) the empty collection kit need not necessarily be the same consumer who transports 90 the empty collection kit. For example, a consumer such as an expectant father or other family member may transport 90 an empty kit that an expectant mother has obtained 80.

After the source collection step 100 of FIG. 2, there may follow one or more of the following steps: a step of source processing into a quantity of valuable biological material suitable for consumer storage (such as step 200 in FIG. 1); a step of consumer storage of the valuable biological material (such as step 300 in FIG. 1); and/or a step of consumer transport of the in-use kit; etc., with consumer storage being a highly preferred step. The steps 80, 90, 100, 200, and 300 on FIGS. 1 and 2 may be combined in various combinations, including omitting one or more of the mentioned steps, for consumer-oriented methods and systems according to the present invention.

When consumer-take-away of valuable biological material is practiced according to the present invention, as compared to a conventional process in which the consumer is uninvolved in collecting the stem cells, cord blood or other material to be stored, there may be shifted responsibility onto a consumer who generally is most highly motivated to ensure collection, of the materials from the correct baby. Consumers may prefer to accept personal responsibility for take-away of valuable biological material to be processed and stored, rather than wondering if strangers entrusted with the responsibility are duly performing their duties. The present invention provides for consumers (such as expectant parents and other family members) to take personal responsibility for duly collecting, in the delivery room or other place where the baby is delivered, the valuable biological material from the baby to whom the consumer is related. The present invention makes possible the placing of responsibility for proper collection (such as collecting materials of the proper baby, etc.) and/or proper storage (such as, e.g., storage of a placenta, umbilical cord, cord blood or of any sample derived from any of a placenta, umbilical cord, cord blood (such as a sample of stem cells, etc.), on at least one individual who may be viewed as most highly motivated for correctly accomplishing proper collection and/or proper storage.

When consumer-storage is practiced is according to the present invention, a consumer and/or his or her family may reduce or eliminate reliance on others (such as a stem cell bank, etc.), and/or increase the amount of control and influence that an individual and/or the family of the individual may have over his, her or their health, appearance, and/or medical condition.

The invention also provides for consumer-storage of valuable biological material for reducing insurance rates. Namely, an individual for whom there is stored valuable biological material (such as stem cells, etc.) that would be medically useful, if necessary, for treating the particular individual if he or she should develop certain diseases or conditions (such as cancer, etc.) is more easily cured or treated upon developing certain diseases or conditions than an individual for whom no such stored stem cells or the like are available. When evidence of storage of such pertinent valuable biological material can be provided, it may be possible to obtain reduced insurance rates for a benefitted individual. Consumer-storage of valuable biological material according to the present invention may provide such insurance cost-reducing advantages.

Additional inventive examples are given below, without the invention being in any way limited thereto.

### EXAMPLE 1

A collection kit is provided by an Ob/Gyn physician to a consumer who is a parent-to-be. The collection kit includes a compartment for receiving a collected placenta, umbilical cord and/or cord blood of a baby being born to the consumer. The collection kit accompanies the consumer to the delivery of the baby, and is used for collecting at least one of the placenta, umbilical cord and/or cord blood sample in the compartment of the collection kit.

### EXAMPLE 2

The collection compartment from Example 1 is carried by the consumer to a location where the collected material (placental, umbilical cord and/or cord blood sample) are treated to extract desired stem cells and there is prepared therefrom a quantity of stem cells of the child, including such reagent(s) and treatment(s) to enhance storability. The storable quantity of stem cells of the child is provided to the consumer for long-term storage.

### EXAMPLE 3

The consumer stores the storable quantity of stem cells of the child in a storage container which is portable.

### EXAMPLE 4

The consumer stores the storable quantity of stem cells of the child in a non-portable, safe-like storage container.

It will be appreciated that variations and modifications from the embodiments set forth above may be made without departing from the spirit of the invention, and that such modifications are within the present invention.

1. A system for consumer control of valuable biological material, comprising at least the steps of:
A) collection of a source of valuable biological material of an infant or young child;
B) processing the source of valuable biological material into a quantity of valuable biological material suitable for consumer storage.

2. The system of claim 1, further including a step C) of consumer storage of the quantity of valuable biological material from step B).

3. The system of claim 2, wherein the quantity of valuable biological material under consumer storage is being stored under direct physical custody and control of at least one parent, or parent's designated guardian or custodian, of the infant or young child from whom collection was undertaken.

4. The system of claim 2, wherein the quantity of valuable biological material suitable for consumer storage is in a container labeled with identifying information and/or instructions.

5. A storage device for consumer storage of a quantity of valuable biological material of a human child, comprising:
a compartment into which may be received the valuable biological material having certain desired biological features;
a fastening system for securing the compartment containing the valuable biological material to provide a secured, closed compartment housing the valuable biological material; and
a preservation system for maintaining the certain desired biological features of the valuable biological material in the secured, closed compartment.

6. The storage device of claim 5, wherein the device is one or more of: (A) portable and easily carried by one individual; (B) useable for self-storage by at least one parent of umbilical cord material of a child of the parent.

7. The storage device of claim 5, wherein the valuable biological material is from a human umbilical cord.

8. The storage device of claim 5, wherein a value of the valuable biological material is for actually or potentially treating or alleviating a medical condition, disease, disorder or problem of the same child.

9. The storage device of claim 5, wherein a value of the valuable biological material is for actually or potentially effecting a cosmetic result for the same child.

10. The storage device of claim 5, wherein the child from whom the valuable biological material came was a newborn human, and wherein the valuable biological material has been stored and preserved since at least one of: infancy of the child; the child being a toddler; the child becoming a teenager; the child surviving teenage years; the child surviving his or her twenties; the child surviving his or her thirties; the child himself or herself having a baby.

11. A system for maintaining physical control by a consumer of valuable biological material, comprising at least one of:
A) direct consumer responsibility for and/or direct consumer supervision of collection of a source of valuable biological material of an infant or young child related to the consumer; and/or
B) direct consumer storage of the source of valuable biological material and/or a quantity of valuable biological material derived from the source of valuable biological material.
